(19) United States
(12) Patent Application Publication
(10) Pub. No.: US 2018/0200373 A1
(43) Pub. Date: Jul. 19, 2018

(54) ILEUM-TARGETING, MUCOADHESIVE THIOLATED HPMCP VACCINE PROTEIN DELIVERY AGENT

(71) Applicant: SEOUL NATIONAL UNIVERSITY R&DB FOUNDATION, Seoul (KR)

(72) Inventors: Chong Su Cho, Seoul (KR); Yun Jaie Choi, Gyeonggi-Do (KR); Sang Kee Kang, Seoul (KR); Bijay Singh, Seoul (KR); Maharjan Sushila, Seoul (KR)

(21) Appl. No.: 15/742,792

(22) PCT Filed: May 13, 2016
(86) PCT No.: PCT/KR2016/005122

§ 371 Date: Jan. 8, 2018

(30) Foreign Application Priority Data
Jul. 8, 2015 (KR) 10-2015-0097285

(51) Int. Cl. A61K 47/38; A61K 39/02

(52) U.S. Cl. CPC A61K 47/38; A61K 2039/55583; A61K 39/02

(57) ABSTRACT

The present disclosure relates to a thiolated hydroxypropyl methylcellulose phthalate (T-HPMCP) drug delivery vehicle which is pH responsive and is loaded with either a protein drug or an antigen, to T-HPMCP microparticles, and to a production method for an ileum-specific, pH responsive, T-HPMCP drug delivery vehicle, the method including a step of loading a protein drug or an antigen onto the T-HPMCP microparticles. The T-HPMCP microparticles of the present disclosure are soluble in chlorinated methane because of the introduction of the thiol group, while a T-HPMCP drug delivery vehicle produced from the particles can efficiently effect in vivo delivery of the protein drug or antigen which is loaded thereon, since said vehicle is pH responsive such that the in vivo residence time is extended and the vehicle can act specifically on the ileum.

### TECHNICAL FIELD

The present disclosure relates to a thiolated hydroxypropyl methylcellulose phthalate (T-HPMCP) drug delivery vehicle which is ileum-specific pH responsive and is loaded with either a protein drug or an antigen. Also disclosed is a method for producing T-HPMCP microparticles, in which the method includes homogenizing thiolated hydroxypropyl methylcellulose phthalate in the presence of an organic solvent. Further, the present disclosure relates to a method for producing a T-HPMCP drug delivery vehicle which is ileum-specific pH responsive, in which the method includes loading a protein drug or an antigen in the T-HPMCP microparticles.

### BACKGROUND ART

Effective delivery of orally administered proteins to the body must overcome several physiological barriers such as low pH, degradation by enzymes, short delivery times, uncontrolled release, and low uptake by microfold cells (M cells). In particular, the difficulty of protein delivery due to different pHs in the gastrointestinal tract, such as stomach, jejunum, duodenum and ileum, is widely known.

Among them, ileum, which refers to the end portion of the small intestine leading to the duodenum and jejunum, is known to have a difficulty in delivering the drug orally administered at its site and have a higher pH environment than other gastrointestinal tracts.

Thus, the development of a pH-responsive delivery agent that is capable of selectively delivering the loaded drug without being dissolved at a low pH is important for efficient protein delivery in the ileum, but despite its importance, such a commercially available delivery agent has not been developed yet.

Among the enteric agents, capsules and tablets are coated with a special coating, which remains on the stomach, and the exposure of the ingredients in the small intestine is referred to as “enteric coating.” The materials used for enteric coating of tablets and capsules include fat, fatty acids, wax, shellac, cellulose acetate phthalate, and the like. Among various enteric coating materials, hydroxypropyl methylcellulose phthalate (HPMCP) is used as an enteric coating agent for tablets and capsules, and is produced by chemical synthesis method using natural pulp as a raw material (KOREAN J. FOOD SCI. TECHNOL. Vol. 44, No. 2, pp. 168-172, 2012).

The HPMCP is widely used as a preparation for oral administration, but the delivery of protein delivery using HPMCP is lowered due to the solubility of HPMCP dissolved at pH 5.5 near the pH of the duodenum.

On the other hand, usually, most of the orally administered delivery agents only deliver drugs directly through the gastrointestinal tract and do not adhere to gastric mucosa or transport through gastric mucosa. The resulting short delivery time cannot transfer encapsulated drugs sufficiently, resulting in a reduced drug efficacy of the loaded drug. Thus, the preparation of drug delivery agents with high mucoadhesive properties is an important task that can improve protein delivery.

### DISCLOSURE

### Technical Problem

The present inventors have made efforts to produce a drug delivery vehicle having mucoadhesive property with an ileum-specific pH response, and as a result, the present disclosure has been completed by producing an ileum-specific protein delivery agent using thiolated HPMCP.

### Technical Solution

An object of the present disclosure is to provide a thiolated hydroxypropyl methylcellulose phthalate (T-HPMCP) drug delivery vehicle which is ileum-specific pH responsive and is loaded with either a protein drug or an antigen. Also, an object of the present disclosure is to provide a method for producing T-HPMCP microparticles, in which the method includes homogenizing thiolated hydroxypropylmethyl methylcellulose phthalate in the presence of an organic solvent, and a method for producing a T-HPMCP drug delivery vehicle which is ileum-specific pH responsive, in which the method includes loading a protein drug or an antigen in the T-HPMCP microparticles.

### Advantageous Effects

The T-HPMCP microparticles of the present disclosure have solubility in methane chloride by the introduction of a thiol group, and the T-HPMCP drug delivery vehicle prepared from the microparticles has a pH response, thereby prolonging the residence time in the body and acting specifically in the ileum so that the delivery of the loaded protein drug or antigen to the body can be efficiently carried out.

### DESCRIPTION OF DRAWINGS

FIG. 1 is a schematic diagram of an oral delivery agent of a vaccine targeting M cells in the ileum. This figure indicates intraluminal pH and GI (gastrointestinal) transport times (distances not expressed to scale). The microparticle (MP) was expected to begin to dissolve in the ileum for the ingestion of antigen released via M cells.

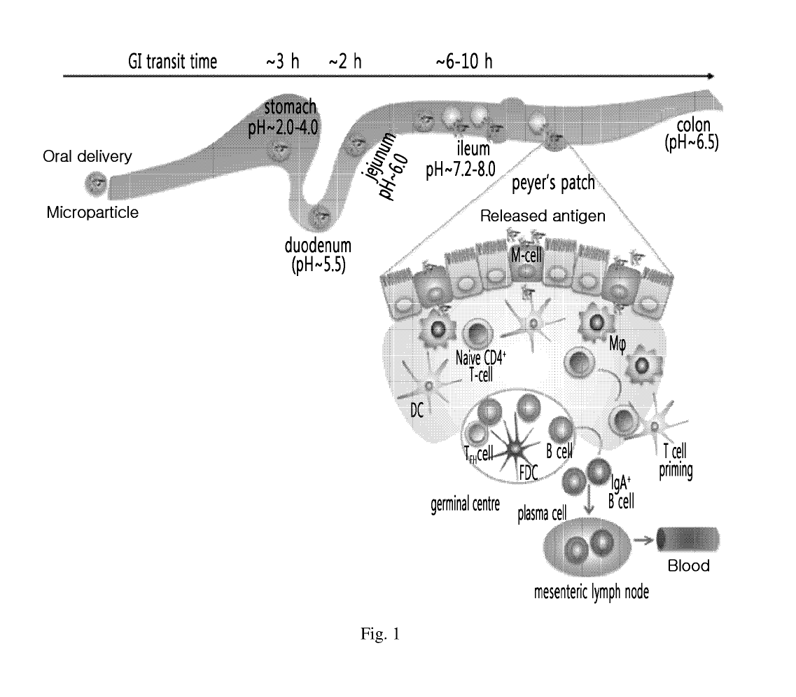

FIG. 2 is a schematic diagram of the oral immunization schedule. Each group of mice was given 6 doses (2 primings and 4 boosts) of antigen. To observe the immune response, serum and excrement samples were collected as outlined in the schematic diagram.

FIG. 3 is a diagram illustrating the reaction for the synthesis of T-HPMCP.

FIG. 4A is a diagram for confirming the synthesis of T-HPMCP using ¹H NMR (DMSO-d₆, 600 MHz). In the NMR spectrum, the conjugation of thiol groups in HPMCP was shown by the proton of —N(H) and —S(H) in cysteine.

FIG. 4B is a diagram for confirming the synthesis of T-HPMCP by FT-IR. N—H bending and stretching corresponding to the spectrum of T-HPMCP appeared.

FIG. 5 is a diagram for analyzing the shape and size of MP. The form of MP was analyzed by SEM (2 μm scale bar). FITC-labeled antigen/MP was observed by CLSM. A. M-BmpB/T-HPMCP MP and FITC-M-BmpB/T-HPMCP MP (Inserted FIG.); C is M-BmpB/HPMCP MP and FITC-M-BmpB/HPMCP MP (Inserted FIG.). The particle size distribution was measured using DLS. B. M-BmpB/T-HPMCP MP; D. M-BmpB/HPMCP MP.

FIG. 6 is a diagram illustrating the release form of M-BmpB released pH-dependently from M-BmpB/T-HPMCP and M-BmpB/HPMCP MP in vitro. Protein-loaded MP (5 mg/ml) was suspended in different pH buffer conditions. The suspended MP was mixed with dichloromethane and stirred at 100 rpm at 37° C. After a given time interval, the amount of released protein in the supernatant was calculated by measuring the uptake at 280 nm. The experiment was performed three times.

FIG. 7 is a diagram illustrating a far ultraviolet circular dichroism spectroscopy before and after being loaded on MP of an antigen. The higher order structure of M-BmpB released from T-HPMCP and HPMCP MP was compared to native M-BmpB.

FIG. 8 is a diagram illustrating mucoadhesiveness analysis of MP in the small intestine. 10 mg of each FITC-labeled MP was dispersed on the intestinal mucosa of each cut pig and cultured at 37° C. for 2 hours with stirring at 100 rpm. The MP attached to the mucosa was removed and hydrolyzed with NaOH, and the absorbance of FITC was measured at 495 nm. The experiment was performed three times.

FIG. 9 is a diagram illustrating the localization of FITC-labeled M-BmpB in Peyer's patch of the small intestine of a mouse. A. FITC-labeled M-BmpB/T-HPMCP or M-BmpB/HPMCP MP was orally administered to mice and the localization of the MP was observed using fluorescence microscopy. The green fluorescence signal of FITC-labeled M-BmpB was higher in the Peyer's patch below the FAE area when administered by T-HPMCP MP. B. The intake of FITC-M-BmpB was quantitated by image analysis and normalized to a value of 1.0 for a M-BmpB control group.

FIG. 10 is a diagram illustrating the FITC-labeled antigen uptake of cells with confocal fluorescence microscope images after culturing the dendritic cells with the antigen-loaded MP for 8 hours.

FIG. 11 is a diagram illustrating antigen-specific immune responses after oral immunization with MP. To determine the immune response, serum and excrement samples were collected from mice at weeks 0, 2, and 5 according to the experimental design. Antibody levels were analyzed using ELISA. A. Anti-M-BmpB IgA levels in excrement. B. Anti-M-BmpB IgG levels in serum, C. Anti-M-BmpB IgG1 levels in serum, D. Level of anti-M-BmpB IgG2a in serum are indicated. Statistical significance was compared using M-BmpB alone as a control group (* P<0.05, **P<0.01, and *** P<0.001).

FIG. 12 is a diagram illustrating flow cytometric detection of specific immune cells in the Peyer's patch derived from immunized mice. Peyer's patches were harvested from mice immunized with M-BmpB/T-HPMCP or M-BmpB/HPMCP MP. Immune cells were isolated and stained with CD11c and MHC II markers prior to detection by FACS. The percentage of positive cells was expressed.

FIG. 13 is a diagram illustrating the results of IFN-γ and IL-4 flow cytometry in CD4⁺ T cells. After the final sample was collected, the spleen was aseptically collected from the mice immunized with the antigen. Splenocytes were again stimulated and IFN-γ and IL-4 production of specific immune cells were analyzed by FACS. A comparison of CD4⁺ cells secreting A. CD4⁺IFN-γ⁺ cells; B. CD4⁺IL-4⁺ cells; C. IFN-γ⁺ and IL-4.

FIG. 14 is a schematic diagram illustrating a process of introducing a thiol group into HPMCP using glutathione.

FIG. 15 is a schematic diagram illustrating a process of introducing a thiol group into HPMCP using cysteamine.

FIG. 16 is a diagram illustrating a result of HPMCP FTIR.

FIG. 17 is a diagram illustrating a result of HPMCP-Glutathione FTIR.

FIG. 18 is a diagram illustrating a result of HPMCP-Cysteamine FTIR.

FIG. 19 is a diagram illustrating a result of confirming mucoadhesiveness of HPMCP-Glutathione and HPMCP-Cysteamine.

### BEST MODES OF THE INVENTION

In order to achieve the above object, one aspect of the present disclosure is to provide a thiolated hydroxypropyl methylcellulose phthalate (T-HPMCP) drug delivery vehicle which is ileum-specific pH responsive and is loaded with either a protein drug or an antigen.

Specifically, the hydroxypropyl methylcellulose phthalate drug delivery vehicle may be a drug delivery vehicle, including, but not limited to, L-cysteine, glutathione, or a cysteamine in which a thiol group is introduced and thiolated.

In addition, the drug delivery vehicle of the present disclosure may be dissolved at pH 7.4 or higher, but is not limited thereto.

The term “thiolated hydroxypropyl methylcellulose phthalate (T-HPMCP)” of the present disclosure can be prepared as thiolated HPMCP by a method that can be carried out by those skilled in the art. In one embodiment of the present disclosure, an N, N′-dicyclohexylcarbodiimide (DCC)/N-hydroxysuccinimide (NHS) activated coupling reaction was synthesized, and according to another embodiment, thiolated HPMCP was prepared through reaction with glutathione or cysteamine, but the method is not limited thereto as long as it can introduce a thiol group into HPMCP.

The term “drug delivery vehicle” of the present disclosure means a carrier or diluent which does not inhibit the biological activity and properties of the compound administered without stimulating the organism, and may add and use other usual additives such as antioxidants, buffers and/or bacteriostatic agents if necessary. The drug delivery vehicle is characterized in that it has a pH response that can act specifically on the ileum and an increased mucoadhesiveness.

The drug delivery vehicle of the present disclosure may be microparticles prepared by homogenizing the thiolated HPMCP in an organic solvent, but is not limited thereto. The drug delivery vehicle has a property of dissolving at a high pH, and is particularly excellent in mucoadhesiveness so that it can deliver a substance loaded on an ileum with high efficiency.

The term “T-HPMCP microparticles” of the present disclosure may be microparticles prepared by homogenizing T-HPMCP in the presence of an organic solvent. The average diameter of the T-HPMCP microparticles of the present disclosure may be 0.01 to 1000 μm, particularly 1 to 100 μm, more particularly 1 to 10 μm, but is not limited thereto. The T-HPMCP microparticles are excellent in mucoadhesiveness and can efficiently deliver drugs.

In one embodiment of the present disclosure, T-HPMCP microparticles were prepared using an organic solution in which T-HPMCP was dissolved in dichloromethane. In another example, it was confirmed that the average diameter of microparticles of T-HPMCP was 3.7±0.4 μm (Example 9 and Experimental Example 4).

The term “pH response” of the present disclosure means the property of T-HPMCP dissolved in a pH-dependent manner. Specifically, it can be dissolved at a pH 7.4 or higher near the pH of the ileum. The drug delivery vehicle of the present disclosure has a pH response and is characterized by specifically working on an ileum, which reaches an ileum and dissolves in the ileum, not in the acid stomach, duodenum and jejunum in the process of passing through the digestive tract.

In one embodiment of the present disclosure, the solubility of T-HPMCP in different pH conditions (pH 2.0 to 8.0) was evaluated in consideration of different pHs in the gastrointestinal tract of the body. As a result, it was confirmed that it was not dissolved in an acidic solution having pH 7.0 or lower, whereas it was dissolved only at pH 7.4 or higher (Experimental Example 2).

In another embodiment of the present disclosure, unlike the HPMCP microparticles that showed immediate antigen release at pH 5.5 or higher, the M-BmpB loaded on the T-HPMCP was released at a pH 2.0 at a low level, whereas at pH 7.4, most of the form was released in an intact state (Experimental Example 5).

Thus, the inventors of the present disclosure confirmed that the T-HPMCP drug delivery vehicle of the present disclosure has pH response, so that it can be delivered to the ileum in a state of loading the drug without dissolving of the T-HPMCP before reaching the ileum.

On the other hand, the inventors of the present disclosure confirmed that the drug delivery vehicle of the present disclosure has an effect of improving the mucoadhesiveness in addition to the pH response as described above, and thus it is possible to deliver drugs to be delivered more efficiently.

The term “mucoadhesiveness” of the present disclosure refers to a property that a drug delivery vehicle remains in the digestive tract and can deliver the loaded drug to the body, and the increased mucoadhesiveness increases the intestinal residence time, and increase the body's absorption rate of the loaded protein drug or antigen.

The present inventors have found that regardless of the method of introducing a thiol group, HPMCP with a thiol group exhibits improved mucoadhesiveness, and thiolated HPMCP can efficiently deliver a drug through improved mucoadhesiveness.

HPMCP into which the thiol group of the present disclosure is introduced can form a disulfide bond with the cysteine and thiol groups of mucin of the mucous protein, thereby increasing the mucoadhesiveness of the drug delivery vehicle and enhancing the delivery efficiency of the loaded protein drug or antigen.

Specifically, the antigen may be M-BmpB, but is not limited thereto.

The term “protein drug” of the present disclosure encompasses a protein or peptide or a drug including it as a main ingredient, and can be loaded on the drug delivery vehicle of the present disclosure. The term “protein” which may be included in the protein drug formulations of the present disclosure includes proteins or peptides or analogs, mutants thereof, and the like, which may be naturally occurring, recombinantly engineered or synthetically produced, but are not limited to, those that can have various modifications such as addition, substitution, deletion, or glycosylation of an amino acid or a domain.

The term “antigen” of the present disclosure means all substances capable of inducing an immune response, and examples thereof include proteins, peptides and the like. Specifically, the antigen loaded on the drug delivery vehicle of the present disclosure may be 29.7 kDa of a basic membrane protein B (M-BmpB; pathogenic small intestine spirochaete Brachyspira hyodysenteriae). More specifically, it may be the peptide of SEQ ID NO.: 1, but is not limited thereto as long as it is an antigen capable of being loaded on T-HPMCP.

Specifically, the drug delivery vehicle of the present disclosure may have mucoadhesiveness higher than 1.5 times that of non-thiolated HPMCP.

In addition, the drug delivery vehicle of the present disclosure may remain in the mucosa 50% or higher even after 2 hours of administration.

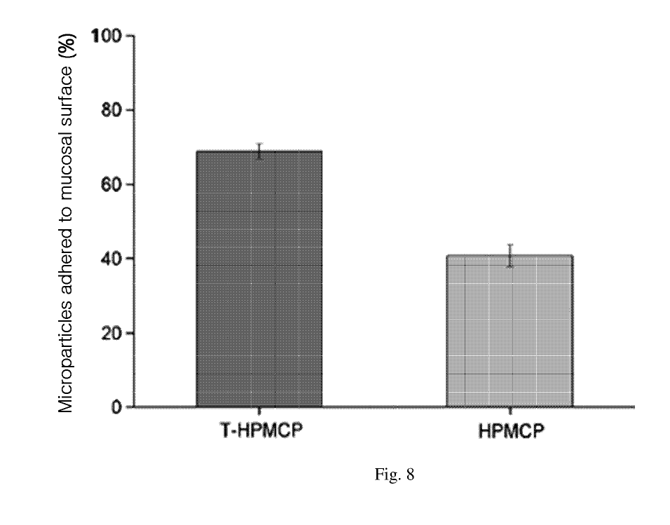

In one embodiment of the present disclosure, the amount of T-HPMCP adhered to the intestinal mucosa of a freshly cut pig was confirmed to have mucoadhesiveness of 1.72 times higher than that of non-thiolated HPMCP (Experimental Example 6 and FIG. 8).

In another embodiment of the present disclosure, when the T-HPMCP drug delivery vehicle was orally administered, the amount of antigen delivered to the Peyer's patch of the ileum was on average of 2.7 times higher than that of the non-thiolized HPMCP drug delivery vehicle (Experimental Examples 7 and 9).

In addition, the drug delivery vehicle of the present disclosure may stimulate CD4⁺ T cells to induce adaptive immunity, and more specifically, the CD4⁺ T cells may produce interferon (IFN)-γ, but is not limited thereto.

The present inventors have loaded the above-mentioned M-BmpB on a drug delivery vehicle to induce an immune response of a mouse. As a result of confirming the delivery efficiency of the antigen, it was confirmed that the immune response was superior to that of the case without thiolization.

Specifically, in the example of the present disclosure, when the antibody was immunized with T-HPMCP, the antigen-specific antibody level was improved as compared with HPMCP, and the rapid increase of CD4⁺ T cells producing IFN-γ was induced (Experimental Examples 9 and

Another aspect of the present disclosure provides a method for producing T-HPMCP microparticles, in which the method includes homogenizing thiolated hydroxypropyl methylcellulose phthalate (T-HPMCP) in the presence of an organic solvent.

Specifically, the organic solvent may be methane chloride, or may be dichloromethane, a mixed solvent of dichloromethane and ethanol, or a mixed solvent of dichloromethane and methanol, but is not limited thereto.

The term “thiolated hydroxypropyl methylcellulose phthalate (T-HPMCP)” of the present disclosure is as described above.

The T-HPMCP of the present disclosure may be different from the conventional HPMCP in solubility in an organic solvent by thiolation. Specifically, the T-HPMCP of the present disclosure can be dissolved in methane chloride. The methane chloride is the most suitable solvent for the production of particles, whereas the conventional HPMCP has a low solubility in methane chloride, but the thiolated HPMCP of the present disclosure has high solubility in methane chloride.

Another aspect of the present disclosure provides a method for producing a T-HPMCP drug delivery vehicle which is ileum-specific pH responsive, in which the method includes loading a protein drug or an antigen on the T-HPMCP microparticles.

The terms “T-HPMCP microparticles,” “protein drug,” “antigen,” “pH response” and “drug delivery vehicle” of the present disclosure are as described above.

The method of loading the protein drug or antigen on the particles can be carried out by a method known to a person skilled in the art.

The T-HPMCP drug delivery vehicle prepared by the method of the present disclosure has a high mucoadhesiveness and has an ileum-specific pH response, and is capable of efficiently delivering drugs to the ileum.

### MODES OF THE INVENTION

Hereinafter, the present disclosure will be described in more detail with reference to the following examples. However, the following examples are for illustrative purposes only and are not intended to limit the scope of the present disclosure.

### Example 1. Preparation of Materials

Hydroxypropyl methylcellulose phthalate-55 (HPMCP) was obtained from Shin-Etsu Chemical Co., Ltd. (Tokyo, Japan). N, N′-dicyclohexylcarbodiimide (DCC), N-hydroxysuccinimide (NHS), L-cysteine hydrochloride monohydrate, dimethyl sulfoxide (DMSO), poly (vinyl alcohol) (PVA), Pluronic® F-127, dichloromethane, 4′,6-diamino-2-phenylindole dilactate (DAPI), carbonate-bicarbonate buffer capsules, fluorescein isothiocyanate (FITC), type VIII collagenase were purchased from Sigma-Aldrich (St. Louis, Mo., USA).

Recombinant mouse granulocyte macrophage colony stimulating factor (GM-CSF) was purchased from Peprotech (New Jersey, USA). Ellman's reagent was purchased from Thermo Scientific (Rockford, USA). His-Bind Resin was purchased from Novagen (California, USA), and Tris-glycine-PAG pre-cast SDS gel was purchased from Komabiotech (Seoul, Korea). α-modified minimum essential medium (α-MEM), RPMI medium and fetal bovine serum (FBS) were purchased from Thermo Scientific HyClone (Waltham, Mass., USA). BD Difco™ LB (Luria-Bertani) broth was obtained from Becton, Dickinson and Company (New Jersey, USA). His-Bind® Resin was purchased from Novagen Inc. (California, USA), and Detoxi-Gel™ endotoxin removing column and bicinchobicinchoic acid (BCA) protein assay reagents (A and B) were purchased from Thermo Scientific Pierce (Illinois, USA).

Horseradish peroxidase (HRP)-conjugated goat anti-mouse IgA, IgG, IgG1 and IgG2a antibodies were purchased from Santa Cruz Biotechnology (Dallas, Tex., USA). BD OptEIA reagent and cytofix/cytoperm solution were purchased from BD Biosciences (California, USA). Ca²⁺/MG²⁺-free (CMF) HBSS buffer was purchased from Life Technologies (MD, USA).

Anti-mouse CD11cAPC, anti-mouse MHC class II-Alexa Fluor 700 and cell stimulation cocktail (including protein transfer inhibitor) were purchased from Ebioscience (CA, USA), while rat anti-mouse (2.4G2) FcγRIII/II, PE Rat anti-mouse IFN-γ, Alexa fluor 488 rat anti-mouse IL-4, and APC rat anti-mouse CD4 were purchased from BD Pharmingen (CA, USA).

### Example 2. Synthesis of Thiolated HPMCP

The thiolated HPMCP (T-HPMCP) of the present disclosure was synthesized through chemical modification of HPMCP using L-cysteine hydrochloride, as shown in the conventional document (Quan J S, Jiang H L, Kim E M, Jeong H J, Choi Y J, Guo D D, et al., pH-sensitive and mucoadhesive thiolated Eudragit-coated chitosan microspheres. International Journal of Pharmaceutics. 2008; 359: 205-10).

Briefly, 4 g of HPMCP is dissolved in 100 ml DMSO and uniformly stirred at room temperature for 24 hours under nitrogenous conditions to allow the carboxylic acid moiety of the polymer to activate with N,N′-dicyclohexylcarbodiimide (DCC, 9 g) and N-hydroxysuccinimide (NHS, 5 g). The byproduct was removed by filtration and the filtrate was reacted with L-cysteine hydrochloride (0.4 g) for 48 hours under similar conditions. The reaction mixture was filtered to remove byproducts and the filtrate was dialyzed against DMSO first, then distilled water to remove unbound L-cysteine. Finally, the product was lyophilized at −20° C. and stored until use. The conjugation of L-cysteine was confirmed by H NMR spectroscopy (Avance 600, Bruker, Germany) and Fourier transform infrared spectrometer (FT-IR; Nicolet 6700 ThermoFisher Scientific Inc., Waltham, Mass., USA).

### Example 3. Determination of Thiol Group of T-HPMCP by Ellman Method

Ellman method was performed according to the manufacturer's instructions to confirm the degree of thiol group substitution in the T-HPMCP prepared in Example 2 above. Briefly, a 10 mg/ml aqueous solution of T-HPMCP was prepared and diluted with 0.1 M sodium phosphate buffer (pH 8) containing 1 mM EDTA to prepare individual dilutions. To each 50 μl aliquot of each dilution was added 500 μl of 0.5 M phosphate buffer (pH 8) and 10 μl of Ellman reagent (DTNB 0.4 mg/ml phosphate buffer 0.5 mg/1, pH 8.0). Control reactions were performed with unmodified HPMCP. Samples were blocked from light and cultured for 15 minutes at room temperature. After 15 minutes, 100 μl of the supernatant was transferred to a microtiter plate and the absorbance of light at a wavelength of 412 nm was measured using an Infinite 200 PRO multimodal reader (Tecan, Switzerland). The amount of free thiol group was calculated from a standard plot obtained by measuring the light absorbance of an aqueous solution of L-cysteine hydrochloride salt monohydrate.

### Example 4. Evaluation of pH Sensitivity

The pH sensitivity of the T-HPMCP of the present disclosure as compared to HPMCP was tested in various buffer solutions of pH 2.0 to 8.0. The polymer at a concentration of 5 mg/ml is immersed in each pH buffer, and particularly 1 mg of T-HPMCP or HPMCP was suspended in 200 μl of potassium hydrogen phthalate buffer (pH 2.0, 3.0 and 4.0), sodium acetate buffer (pH 4.5 and 5.5) or sodium phosphate buffer (pH 6.0, 7.0, 7.2, 7.4 and 8.0).

### Example 5. Expansion Study

Expansion studies were performed with two buffers with different pH systems of simulated gastric juice (pH 1.2) and stimulated intestinal juice (pH 7.4). Initially, a flat 4 mm T-HPMCP or HPMCP disc weighing 30 mg each was prepared. Each disc was immersed in 1.0 mL of each buffer at 37° C. for 6 hours, and the test disc hydrated at the specified time intervals was taken out from the culture buffer, and was weighed with a microbalance immediately after removing the moisture of the surface. Therefore, the swelling degree was determined by gravimetric measurement as follows.

Swelling degree (%)=(Wt−Wo)/Wo*100%

Wt is the weight of the expanded disc at time t, and Wo is the initial weight of the dry disc.

### Example 6. FITC-Labeling of Polymer

Covalent binding of T-HPMCP or HPMCP to FITC was performed as described below. 5 mg of FITC dissolved in 1 mL of DMSO was gradually added to 100 mg of HPMCP dissolved in 2 mL of DMSO:ethanol (2:1) or 100 mg of T-HPMCP dissolved in 2 mL of DMSO. The reaction was carried out in the dark at room temperature for 4 hours and shaken constantly using a Rotating Shaker (FINEPCR Cp., Ltd., Korea). The reaction mixture was dialyzed with three water changes of distilled water, lyophilized in vacuo and stored at −20° C. until use. The amount of covalently bound FITC was determined by measuring the light absorbance of the FITC-polymer conjugate at 455 nm based on the standard curve.

### Example 7. Isolation and Purification of Model Protein Antigen

M cell-homing peptide (SEQ ID NO.: 1: CKSTHPLSC) linked to a gene expressing the M-BmpB protein, namely BmpB (a 29.7 kDa outer membrane lipoprotein of the pathogenic small intestine spirochaete Brachyspira hyodysenteriae) was seeded in 4 ml of LB medium supplementing a single E. coli colony contained therein with 100 μg/ml of ampicillin and shaken cultured overnight at 37° C. 500 μl of seed medium was used to inoculate 800 ml of the same medium supplemented with 100 μg/ml ampicillin and cultured at 37° C. with shaking at 200 rpm. When the cultured tissue reached an OD₆₀₀ of 0.5 to 0.7, protein expression was induced using 1 mM IPTG and culturing was continued for 12 hours. Cells were recovered by centrifugation at 6000×g for 10 minutes, washed twice with ice-cold phosphate buffered saline (PBS), and resuspended in 25 ml histidine binding buffer. Then, it was cultured on ice for 10 minutes. Cells were sonicated for a total of 8 minutes in a cycle of 9 second pulses and 4 second atmosphere in an ice bath (Vibra Cell; Sonics & Materials, Newtown, USA). Some of the soluble lysates were removed by centrifugation at 4° C. for 30 minutes at 12,000×g. The degree of protein expression was confirmed on 4 to 20% SDS gels using sodium dodecyl sulfate polyacrylamide gel electrophoresis (SDS-PAGE).

Some histidine-labeled soluble proteins were purified using His-Band® Resin according to the manufacturer's instructions. Briefly, soluble protein extracts were loaded into His-Bind®Resin (5 ml) and equilibrated to a 12 column volume of histidine-binding buffer (5 mM imidazole, 0.5 M sodium chloride, 20 mM tris-Cl, pH 7.9), and charged with a charging buffer (50 mM nickel sulfate). Followed by washing with histidine-binding buffer and washing again with wash buffer (10 mM imidazole, 1 M sodium chloride, 20 mM tris-Cl, 8.7% glycerol, pH 7.9). Proteins were eluted using elution buffer (200 mM imidazole, 20 mM tris-Cl, pH 7.9). The eluted portion was analyzed by 4-20% SDS-PAGE and then stained with Coomassie Brilliant Blue R-250. The purified histidine-labeled protein was dialyzed into water (pH 7.9), which was replaced three times at 4° C. for 24 hours. Endotoxin was removed using Detoxi-Gel™ Endotoxin to remove the column as directed by the manufacturer. Protein purity was determined by SDS-PAGE. The protein concentration was determined by measuring the absorbance at 280 nm using a Nanophotometer (Implen GmbH, Germany). The purified proteins were lyophilized and stored at −20° C. until use.

### Example 8. FITC-Labeling of Protein Antigen

1 mg FITC dissolved in 200 μl DMSO was gradually added to 20 mg of M-BmpB protein dissolved in 2 mL carbonate-bicarbonate buffer and the reaction mixture was shaken cultured constantly for 4 hours at room temperature in the dark using Rotating Shaker. The reaction mixture was dialyzed with three water changes of distilled water (pH 8), lyophilized in vacuo and stored at −20° C. until use. The amount of fluorescein covalently bound in FITC-M-BmpB was determined as described above.

### Example 9. Preparation of Microparticles (MP)

### Example 9-1. Preparation of T-HPMCP Microparticles and HPMCP Microparticles

The microparticle (MP) was prepared by a single oil/water emulsion solution evaporation technique. To prepare the organic solution, each of 100 mg of T-HPMCP and HPMCP was dissolved in 5 ml of dichloromethane and dichloromethane:ethanol (25:1), respectively.

The polymer solution was added dropwise to 50 ml of 1% (w/v) PVA and the mixture was homogenized at 11,000 rpm for 4 minutes using an Ultra Turrax (T25, IKA, Germany) to produce an oil-in-water (O/W) emulsion. The emulsion was stirred in a fume cupboard at room temperature for 6 to 8 hours to evaporate the organic solvent. The microparticles (MP) were collected by centrifugation, washed with distilled water and lyophilized in vacuo. The MP was obtained in the form of a white powder and stored at −20° C. until use. FITC-T-HPMCP microparticles and FITC-HPMCP microparticles were prepared in a similar procedure and stored at −20° C. until use.

### Example 9-2. Preparation of MP Loading Antigen

M-BmpB/T-HPMCP or M-BmpB/HPMCP MP was prepared by a water-in-oil-in-water (W/O/W) dual emulsion solvent evaporation method as explained in the conventional document (Singh B, Jiang T, Kim Y K, Kang S K, Choi Y J, Cho C S. Release and Cytokine Production of BmpB from BmpB-Loaded pH-Sensitive and Mucoadhesive Thiolated Eudragit Microspheres. Journal of Nanoscience and Nanotechnology. 2015; 15:606-10).

Briefly, the organic solutions of T-HPMCP and HPMCP were prepared by dissolving T-HPMCP and HPMCP 100 mg in 5 ml of dichloromethane and dichloromethane:ethanol (25:1), respectively. Subsequently, an ultrasonic homogenizer (primary emulsion) was prepared by adding an aqueous phase including 10% Pluronic F-127 solution mixed with 200 μl of water including 5 mg of M-BmpB protein to the solution. The polymer/protein mixture was emulsified using an ultrasonic homogenizer (Sonics, Vibra Cells™) to produce a water in oil emulsion. The mixed emulsion was added to 50 ml 1% (w/v) PVA and the mixture was homogenized for 4 minutes at 11,000 rpm using Ultra Turrax (T25, IKA, Germany) to prepare a W/O/W emulsion. The emulsion was stirred at room temperature for 6 to 8 hours on the ventilation wall to evaporate the organic solvent. The MP loading the antigen produced therefrom was collected by centrifugation, rinsed with distilled water, and lyophilized in vacuum. Antigen-loaded MP was obtained in the form of a white powder and stored at −20° C. until use. Similarly, FITC-M-BmpB/T-HPMCP MP and FITC-M-BmpB/HPMCP MP were prepared and stored at −20° C. until use.

### Example 10. Microparticle Morphology and Particle Size Distribution

The surface morphology and mean size of the microparticles were analyzed by a field-emission scanning electron microscope (FE-SEM) Supra 55VP-SEM (Carl Zeiss, Oberkochen, Germany). Prior to the experiment, the microparticles were mounted on metal stubs with a thin adhesive tape and coated with gold in a vacuum using a coating chamber (CT 1500 HF, Oxford Instrument Oxfordshire, UK). The average diameter and particle-size distribution were measured by dynamic light scattering using DLS-7000 (Otsuka Electronics, Japan).

### Example 11. Loading Antigen (Loading Content) and Encapsulation Efficiency

The amount of antigen encapsulated per unit weight of microparticles (MP) was determined by the extraction method that slightly modifies the method introduced in the conventional document (Carino G P, Jacob J S, Mathiowitz E. Nanosphere based oral insulin delivery. Journal of Controlled Release: Official Journal of the Controlled Release Society. 2000; 65: 261-9).

Briefly, 5 mg of dry particulate was suspended in 250 μl of histidine binding buffer (pH 7.9) and 1.5 ml of dichloromethane was added. To extract the protein from the organic solution into the buffer, the mixture was cultured with constant shaking using a rotating shaker at room temperature for 2 hours. After centrifugation at 6000×g for 10 minutes, 200 μl of aqueous solution was withdrawn and the amount of M-Bmp was calculated using BCA protein assay.

The encapsulation efficiency was expressed as a ratio of the amount of the actually loaded antigen to the total amount of the antigen used for preparation of MP. Each preparation used in the experiment was analyzed three times. The encapsulation efficiency and the loading rate of the antigen were calculated as shown in the following equations.

$$Encapsulation efficiency , \% = \frac{amount of protein in microsperes}{amount of protein initially used} \times 100 \%$$

$$Antigen loading , \% = \frac{amount of protein in microspheres}{amount of microsperes} \times 100 \%$$

### Example 12. Release of In Vitro Antigens from Microparticles

The release of M-BmpB from in vitro M-BmpB/T-HPMCP or M-BmpB/HPMCP MP was evaluated in three physiological buffer conditions of 10 mg/mL (simulated gastric juice (pH 2.0), stimulated intestinal juice (pH 6.0 and 7.4)) and was measured three times by shaking and culturing the microspheres constantly at 37° C. and 100 rpm. The supernatant was collected by centrifugation at 6000×g for 10 minutes according to the predetermined time interval (0, 2, 4, 6, 8, 10, 12 and 24 hours). M-BmpB release amount was quantified using BCA protein assay.

### Example 13. The Structural Integrity of the Antigen Released from the Microparticles

Structural integrity of protein antigens before and after loading in MP was evaluated as circular dichroism (CD). CD measurements were made using a Chirascan™-plus CD Spectrometer (Applied Photophysics Ltd, Leatherhead, UK). Far-UV CD spectra were measured with Quartz cuvettes (0.1 cm path length) at 0.5 second sampling time per 1 nm wavelength in the range of 260 to 200 nm.

### Example 14. Mucoadhesiveness of Extracellular Microparticles

To evaluate the mucoadhesiveness of MP prepared in Example 9, 10 mg of each of FITC-T-HPMCP MP and FITC-HPMCP MP suspended in 1 ml sodium phosphate buffer (pH 7.0) and sodium acetate buffer (pH 5.0) was applied to the small intestinal mucosa of freshly excised pigs fixed on a slide. The slides were then vertically placed in a falcon tube including each of the 40 ml pH buffers described above and cultured for 2 hours at 37° C. with shaking at 100 rpm. The mucosa-adhered microparticles were removed and hydrolyzed with sodium hydroxide (1M) for 30 min at 37° C. Samples were centrifuged at 10,000×g for 5 minutes and 200 μl of the supernatant was transferred to a microplate reader. The absorbance of FITC was measured at 495 nm and the concentration was measured by interpolation method from a standard curve. The experiment was repeated three times.

### Example 15. Antigen Delivery Efficiency of the Body Microparticles

The efficiency of antigen delivery by T-HPMCP MP was evaluated by protein antigen uptake by M cells in FAE of Peyer's patch. FITC-M-BmpB/HPMCP MP and FITC-M-BmpB/T-HPMCP MP equivalent to 200 μg of encapsulated protein were injected into mice (7 weeks old Balb/c, 20 g). Eight hours after oral administration, the mice were euthanized and a portion (˜2 cm) of the intestine including the Peyer's patch was cut, and then extensively washed with cold PBS and fixed with formalin.

For cryo-sectioning, tissue samples were placed in the optimal cutting temperature medium and sections of the frozen tissue (10 μm thick) were cut on a Leica CM1850 cryomicrotome (Leica Microsystems Inc., USA). The tissue sections were air-dried, stained with DAPI in −20° C. acetone, and visualized under a confocal laser scanning microscope (CLSM). As shown in the conventional document (Knoop K A, Kumar N, Butler B R, Sakthivel S K, Taylor R T, Nochi T. et al., RANKL Is Necessary and Sufficient to Initiate Development of Antigen-Sampling M Cells in the Intestinal Epithelium. J Immunol. 2009; 183:5738-47), the quantitative analysis of protein antigen uptake by M cells in the Peyer's patch was performed using ImageJ v1.36b software (http://rsb.info.nih.gov/ij/).

### Example 16. Intake of Antigen Released from Microparticles of Dendritic Cells

JAWS II, a murine dendritic cell line, was supplemented with 20% FBS, 5 ng/mL GM-CSF, 100 U/ml penicillin G, and 100 ug/ml streptomycin in α-MEM including ribonucleoside and deoxyribonucleoside and stored at 37° C. in an atmosphere of 5% CO2. Cells were seed-cultured in 35 mm glass-bottomed dishes (2×10⁵ cells/dish) for 48 hours. The cells were treated with FITC-labeled M-BmpB/T-HPMCP MP or FITC-labeled M-BmpB/HPMCP MP 200 μg/well and was cultured for 8 hours at 37° C. The medium was aspirated and the cells were washed with PBS. Cell uptake of FITC-labeled M-BmpB released from MP was analyzed by confocal laser scanning microscope (CLSM) LSM 510 (Carl Zeiss, Germany).

### Example 17. Oral Immunization of a Mouse

Five BALB/c female mouse group of 6 weeks old was used for the experiment. The mice were purchased from Samtako, Co. Ltd. (Osan, Korea) and placed in a cage under standard aseptic conditions along the guideline for the use of experimental animals (Seoul National University). The mice were randomly fed and watered. After a week of acclimatization, mice were immunized with oral gavage using a 1 ml syringe suitable for oral ingestion needles and mice were immunized with 200 μl of MP equivalent to 200 g of protein suspended in an appropriate buffer. Each group of mice received a total of 6 vaccines (2 primings and 4 boosts). Priming was administered on days 0 and 1, and booster immunization was performed on days 7, 8, 14, and 15. A naked mouse group inoculated with PBS and washed with M-BmpB solution was used as a control group. The same dose was used for priming and booster immunization.

### Example 18. Sampling of Blood and Excrement

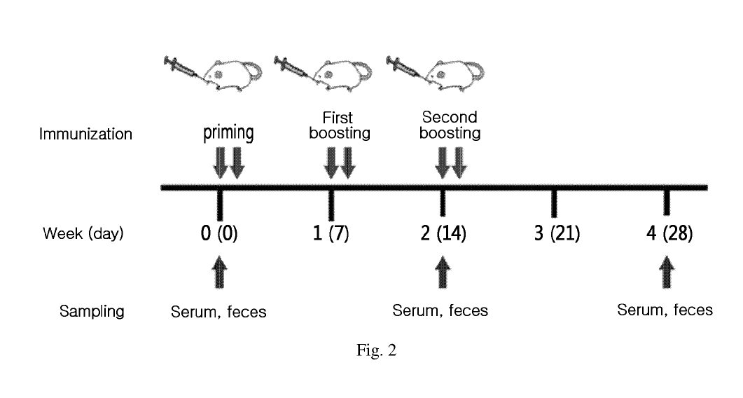

Blood samples of animals immunized from the tail vein were collected three times before immunization, two weeks after primary immunization and two weeks after final booster immunization. Serum from blood coagulation samples was centrifuged at 3,000×g for 10 minutes and used for the detection of antigen-specific antibodies by ELISA. Similarly, feces from immunized animals were collected three times at the same time as the blood samples (FIG. 2). The fecal pellet were homogenized in 5 volumes of PBS at 4° C., centrifuged at 6,000×g for 10 minutes, and the supernatant was collected and analyzed for the presence of antigen-specific IgA by ELISA. After the last sampling, the mice were euthanized and dissected to detect specific immune cells by fluorescence-activated cell sorting (FACS) analysis to isolate the Peyer's patch from the ileum and spleen.

### Example 19. Antigen-Specific Antibody Detection by ELISA

The levels of serum M-BmpB specific immune globulin antibodies G (total IgG) and selected IgG isotypes (isotype, IgG1 and IgG2a) and the levels of M-BmpB specific IgA in fecal samples were determined by ELISA using a BD OptEIA kit (BD Biosciences, California, USA) according to a manufacturer's instruction. Briefly, M-BmpB protein antigen (25 μg/ml) was diluted with carbonate buffer (pH 9.6) and diluted antigen was used to coat wells (100 μl/well) of polystyrene microtiter plates. The plates were cultured overnight at 4° C. The plate was then washed with wash buffer and blocked with 200 μl/well of assay diluted solution for 1 hour at 37° C. Following 37° C. blocking, sera from mice diluted 1:3000 in assay diluted solution were added to the wells (100 μl/well). Feces samples were diluted 1:100. All samples were tested three times.

For specific antibody detection, the plates were cultured with appropriately diluted HRP-labeled goat anti-mouse immunoglobulin antibody conjugate specific for IgG, IgG1 and IgG2a (1:5000 dilution) or IgA (1:2000 dilution) for 1 hour at room temperature. The plate was washed three times with wash buffer and treated with a substrate solution of 100 μl/well in the dark for 30 minutes. Then, 100 μl/well of stop solution was added to stop the enzyme reaction. Ultimately, the absorbance was measured with an Infinite 200 PRO multimode microplate reader at 450 nm.

### Example 20. Isolation of Immune Cells from Ileum's Peyer's Patch

After final sampling from the immunized mice, the mice were dissected to obtain a Peyer's patch from the ileum. Furthermore, immune cells were isolated as described in the conventional document (Geem D, Medina-Contreras O, Kim W, Huang C S, Denning T L. Isolation and characterization of dendritic cells and macrophages from the mouse intestine. Journal of Visualized Experiments: JoVE. 2012: e4040).

Briefly, a portion of the small intestine containing the Peyer's patch was cut longitudinally and the epithelial layer was removed by three consecutive 15 minute cultures at 2 mM EDTA in 37° C. CMF HBSS buffer. The tissue was digested with 1.5 mg/ml Type VIII collagenase in CMF HBSS/FBS. After passing through a 100 μm cell filter, the cell suspension was centrifuged at 1500 rpm for 5 minutes at 4° C. The cells were washed twice in ice-cold CMF PBS and blocked for 10 minutes on ice with 2.4G2 anti-FcγRIII/II antibody in cold staining buffer (CMF PBS+5% FBS). After washing with cold staining buffer, the cells were stained with antibody staining cocktail (CD11c and MHC class II) on ice for 20 minutes in the dark. Finally, the cells were washed twice with cold staining buffer and resuspended in 400 μl of very cold staining buffer for FACS analysis.

### Example 21. Detection of Flow Cell Counts of IFN-γ and IL-4 in CD4⁺ T Cells

After immunization with MP-immunized mice, the spleen was aseptically obtained and a single cell suspension of spleen cells was prepared in RPMI supplemented with 10% heat-inactivated FBS. ACK lysis buffer was used to dissolve RBCs and spleen cells (2×10⁶ cells per well) were seeded in 96 well round-bottom plates. After 16 hours of stimulation with cell-stimulating mixture (including protein transport inhibitor), cells were collected by centrifugation at 2,000 rpm for 5 minutes and washed twice with PBS. Cells were fixed and permeabilized in a cytofix/cytoperm solution for 20 minutes at 4° C. in the dark and stained with cell-specific (CD4⁺) and intracellular cytokine-specific (IFN-γ and IL-4) antibody. Finally, stained cells were analyzed by FACSCalibur (Becton Dickenson, USA).

### Example 22. Statistical Analysis

All results were expressed as mean±standard deviation. Statistical significance was tested using a unidirectional distribution (ANOVA) and then a least significance test was performed. Statistical significance was expressed as * P<0.05, ** P<0.01, and *** P<0.001.

The results of the experiments of the above Examples were analyzed as in the following Experimental Examples.

### Experimental Example 1. Synthesis and Characterization of T-HPMCP

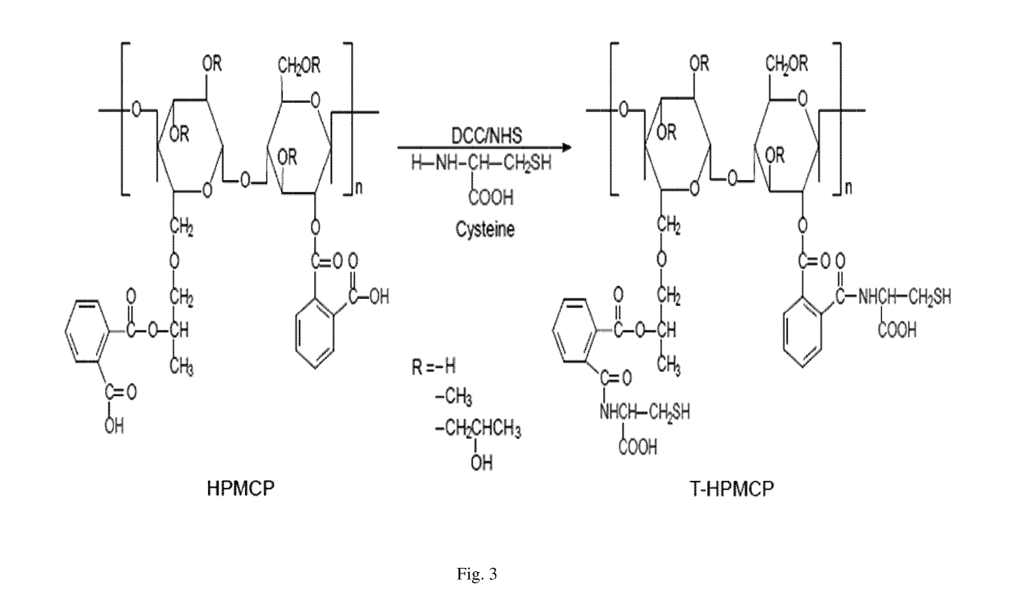

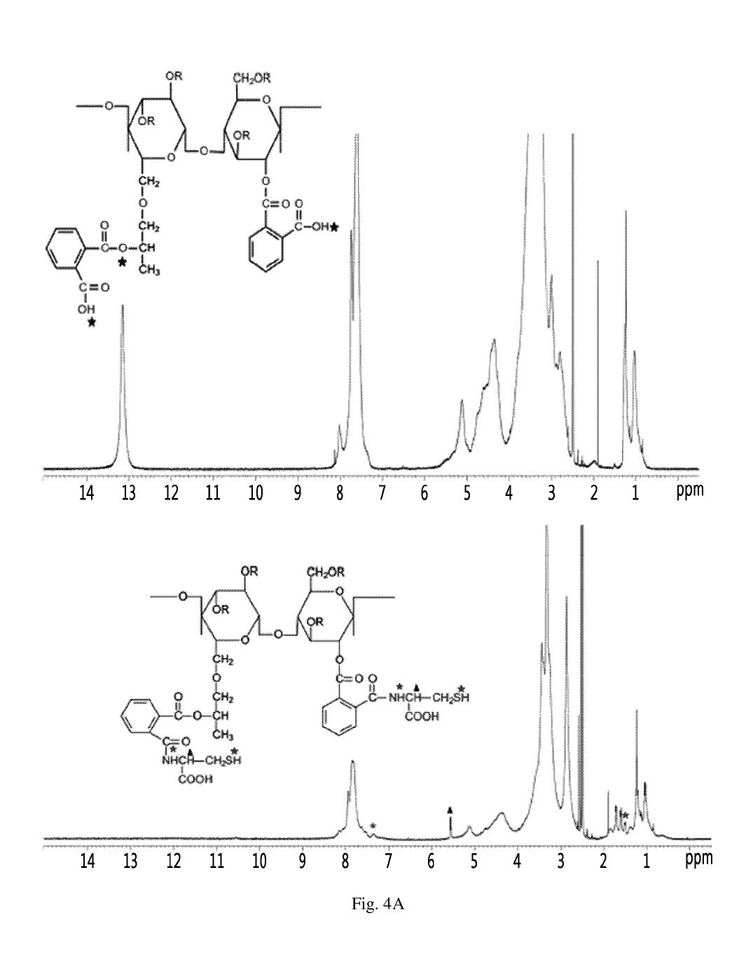

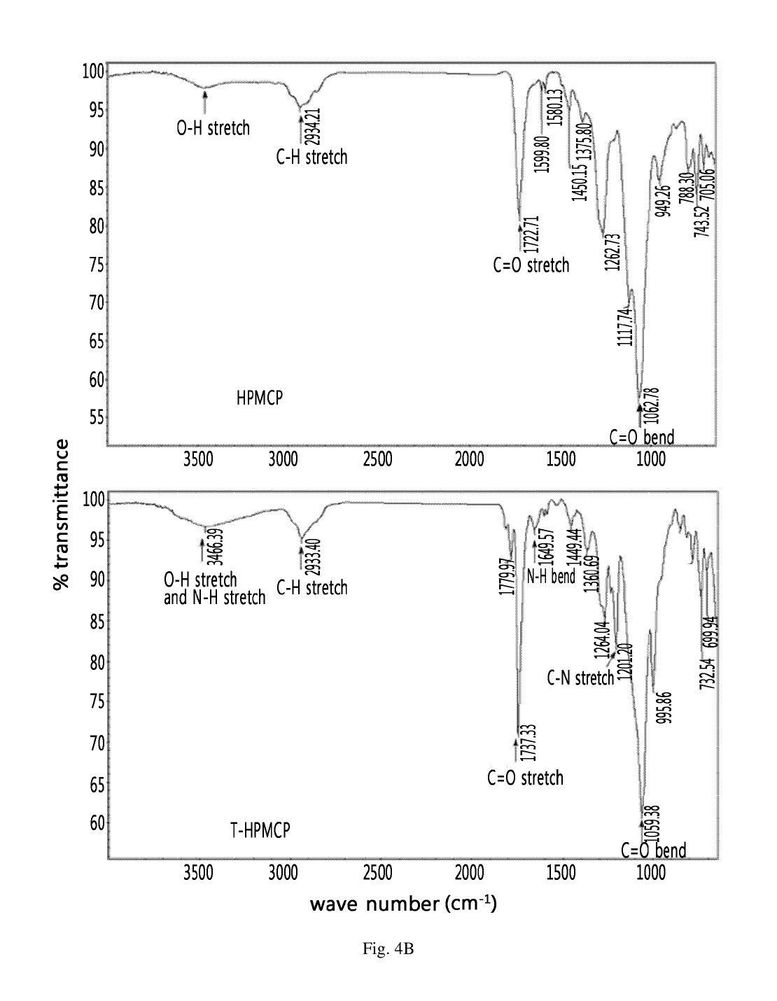

T-HPMCP was synthesized by DCC/NHS activated coupling reaction as illustrated in FIG. 3. Coupling of cysteine and HPMCP was confirmed by proton nuclear magnetic resonance (¹H-NMR) and Fourier transform infrared spectroscopy (FT-IR). The peak of amide and thiol protons appeared in the ¹H-NMR spectrum of T-HPMCP. The weaker peak appeared at 7.4 ppm, which is consistent with the contribution of the amide proton. Also, the thiol proton resonance showed a strong peak at 1.6 ppm (FIG. 4A). The cysteine conjugate of T-HPMCP was confirmed by peaks newly shown at 1649 cm⁻¹ and 1201 cm⁻¹ of FT-IR spectrum, and each peak corresponds to NH bending vibration and CN stretching mode (FIG. 4B). The FT-IR spectrum of the T-HPMCP spectrum additionally showed the characteristic peaks of amide bonds including 1737 cm⁻¹ C═O stretching vibration, 1059 cm⁻¹ C═O bending vibration and 3466 cm⁻¹ N—H stretching vibration. The thiol content of T-HPMCP was 15.5 mol-%.

### Experimental Example 2. pH-Sensitivity of T-HPMCP

The solubility of T-HPMCP in consideration of different pH per each part of the gastrointestinal tract (GI tract) such as stomach (pH 2.0 to 4.0), duodenum (pH 5.5), jejunum (pH 6.0) and ileum (pH 7.2 to 8.0) was evaluated in the range of pH 2.0 to 8.0. Unlike HPMCP, which dissolves completely at pH 5.5 or higher, T-HPMCP was not dissolved in acidic solutions at pH 7.0 or lower but dissolved only at pH 7.4 or higher.

Similarly, the expansion of the T-HPMCP disc was compared to HPMCP discs that were not unmodified at pH 2 and 4. The T-HPMCP disc showing a swelling rate of 90.42±6.5% for 1 hour at pH 7.4 and an expansion rate of 50.64±1.5% for 1 hour at pH 2.0 had higher water absorption capacity at both pHs.

HPMCP discs were completely degraded at pH 2.0 within 1 hour and completely dissolved at pH 7.4 while T-HPMCP discs were degraded at a slow and constant rate after 2 hours of culturing at both pH 2.0 and 7.4.

### Experimental Example 3. Protein Loading Efficiency of T-HPMCP

The model protein antigen (M-BmpB) was used to evaluate the oral administration efficiency of T-HPMCP protein. A dual-emulsion method was used to encapsulate M-BmpB into T-HPMCP and HPMCP in the form of microparticles (MP).

As a result, T-HPMCP MP exhibited an encapsulation efficiency of 83.20±1.43% loading 7.54±1.71% antigen, whereas HPMCP MP exhibited an encapsulation efficiency of 80.97±1.55% loading 2.86±1.32% antigen.

### Experimental Example 4. Morphology and Size of T-HPMCP Microparticles

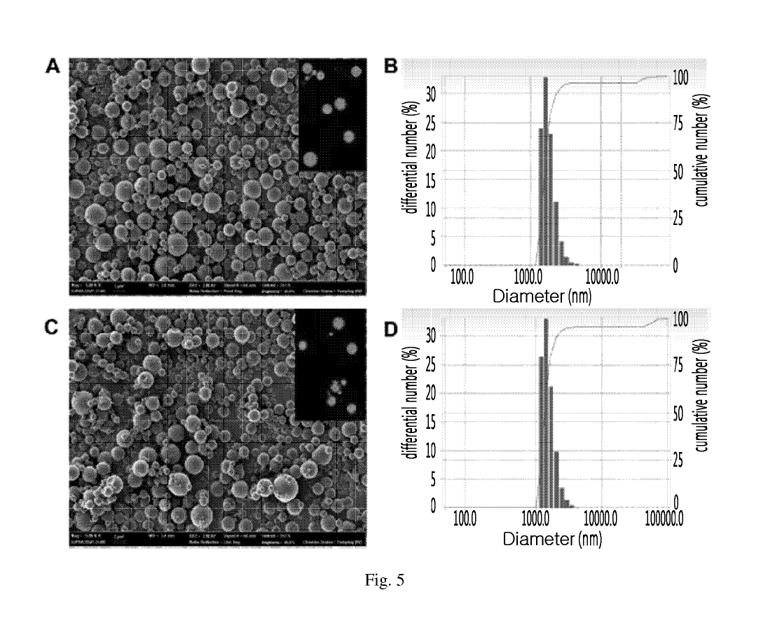

SEM was used to study morphology of MP. Both MPs were well formed with spherical particles with smooth surfaces (FIGS. 5A and 5C). Similarly, when FITC-labeled M-BmpB was encapsulated in T-HPMCP and HPMCP MP and observed with CLSM, it was confirmed as green fluorescence present in MP (FIG. 5).

The size distribution of MP in aqueous solution was measured by DLS. As shown, the average diameter (±SD) of the particles of T-HPMCP and HPMCP were 3.7±0.4 μm and 3.771±0.4 μm, respectively, and had a narrow size distribution (FIGS. 5B and 5D).

### Experimental Example 5. Protein Release and Integrity of T-HPMCP Microparticles

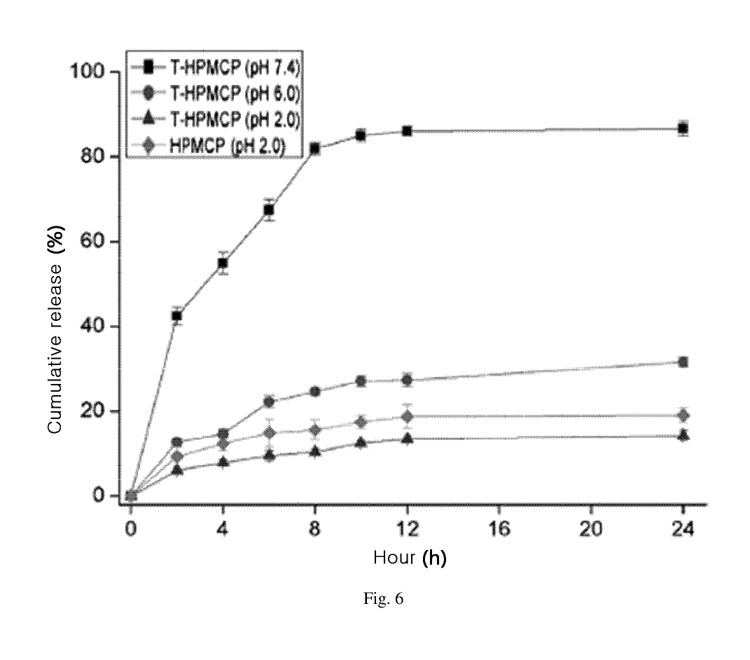

The behavior of M-BmpB released from M-BmpB/T-HPMCP and M-BmpB/HPMCP MP was tested in vitro of environment simulated with stomach (pH 2.0), intestinal (pH 6.0) and ileum (pH 7.4) (FIG. 6). The release form of M-BmpB was expressed as a percentage of the released M-BmpB versus the amount of the encapsulated M-BmpB.

As a result, protein antigen release of T-HPMCP and HPMCP MP was pH-dependent. At pH 2.0, only a small amount of antigen was released from HPMCP MP, but most of the antigens were immediately released from MP at pH 6.0 and 7.4 because HPMCP dissolves at pH 5.5 or higher.

On the other hand, no significant antigen release from T-HPMCP MP occurred at pH 2.0, and slow and controlled release was observed at pH 7.4 after 2 hours of culturing. Approximately 85% of the M-BmpB was released from the T-HPMCP MP within 10 hours.

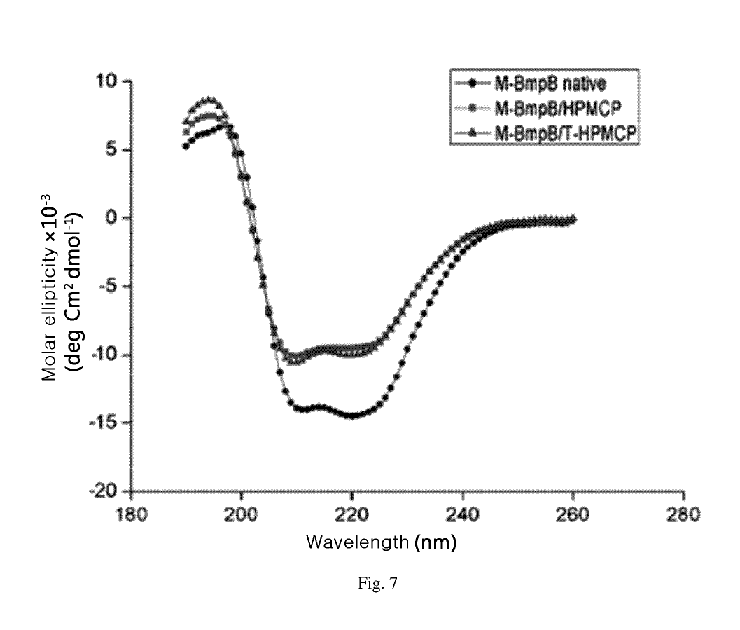

On the other hand, the structural integrity of M-BmpB before and after loading on T-HPMCP and HPMCP MP was evaluated by CD. The CD spectra of M-BmpB released from native M-BmpB and MP are illustrated in FIG. 7. The far UV circular dichroism (far UV-CD) spectrum was consistent with the molar ellipticity minimum value at 223 nm and 210 nm, indicating that the α-helical backbone of M-BmpB released from MP was retained.

### Experimental Example 6. Mucoadhesiveness of T-HPMCP Microparticles

The mucoadhesiveness of T-HPMCP MP was evaluated by using FITC-labeled MP as a fluorescent marker for in vitro experiments using intestinal mucosa of freshly cut pigs. The amount of MP labeled with FITC adhered to the intestine of freshly cut pigs at 37° C. is illustrated in FIG. 8.

From the above results, it was confirmed that 69% of the initially loaded T-HPMCP MP adhered to the mucosa on average while only 40% of HPMCP MP remained on the mucosal surface after 2 hours of culturing. The mucoadhesiveness of T-HPMCP MP was 1.72 times higher than HPMCP MP.

### Experimental Example 7. Ileum-Specific Protein Delivery Efficiency of T-HPMCP MP

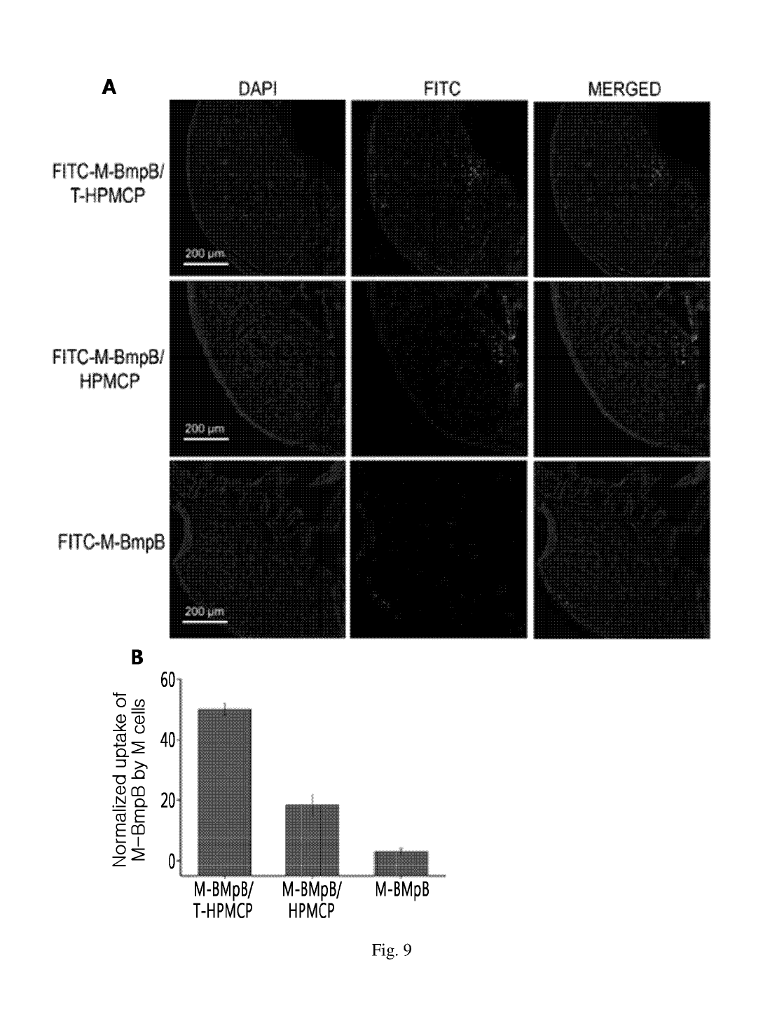

In order to prepare preliminary evidence of selective delivery of the protein in the ileum of T-HPMCP, a proof-of-concept experiment in which FITC-M-BmpB/T-HPMCP or FITC-M-BmpB/HPMCP MP is administered to a mouse in oral gavage was performed. After 8 hours of oral administration, the Peyer's patch of the ileum was cut and frozen. The section of the Peyer's patch was visualized by CLSM (FIG. 9).

As a result, a multitude of antigens that were clearly visible under FAE exhibited an efficient uptake of antigen through M cells. In the form of HPMCP MP, a small amount of antigen passed through the GALT region, whereas when delivered in the form of T-HPMCP MP, the amount of antigen delivered to the GALT region was greater. Furthermore, the antigens were distributed throughout the GALT region when delivered to T-HPMCP MP. When quantified by ImageJ analysis, antigen delivery by T-HPMCP MP was 2.7 times higher than that by HPMCP MP on average.

### Experimental Example 8. Cell Uptake of Antigen Released from T-HPMCP by Dendritic Cells

Cellular internalization of dendritic cells against antigen released from MP was confirmed by in vitro experiments to confirm the efficient delivery of protein antigen by the initiation of immune response of immune cells. JAWS II cells were cultured with FITC-labeled M-BmpB/T-HPMCP or FITC-labeled M-BmpB/HPMCP MP for 4 hours under standard cell culture conditions.

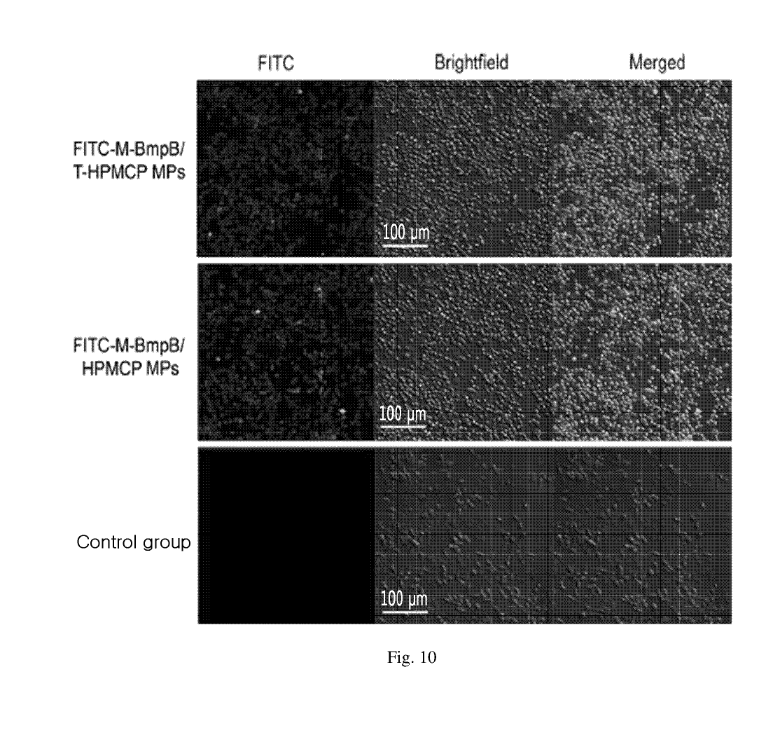

The CLSM image showed that JAWS II cells efficiently ingested FITC-labeled M-BmpB released from T-HPMCP and HPMCP MP (FIG. 10). The cell uptake of antigens released from the MPs was comparable to each other.

### Experimental Example 9. Oral Administration of Protein Antigens Using T-HPMCP Microparticles

In order to induce an immune response to the encapsulated antigen and to confirm efficient protein delivery by T-HPMCP MP, a mouse was immunized with M-BmpB/T-HPMCP MP, M-BmpB/HPMCP MP, M-BmpB, or PBS alone through oral gavage. Serum and fecal samples were collected according to the experimental design and antigen-specific antibodies of the serum and fecal samples were confirmed by ELISA.

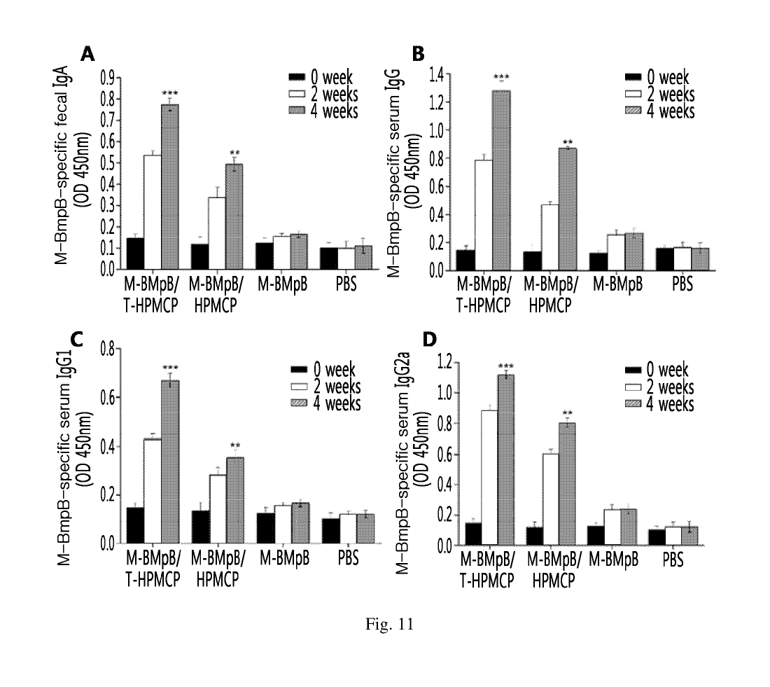

As a result, antigen delivery by T-HPMCP induced a significantly enhanced level of antigen-specific antibody than by HPMCP MP. Immunization of M-BmpB or PBS alone induced negligible immune responses. T-HPMCP MP induced about 1.56±0.20 times higher fecal antibody levels (FIG. 11A) and about 1.63±0.21 times higher serum antibody levels (FIG. 11B) than with HPMCP MP.

In comparison of immunization with M-BmpB alone, it induced 4.66±0.18 times higher fecal antibody level and 4.78±0.12 times higher fecal antibody level when immunized with T-HPMCP MP.

Similarly, serum samples were analyzed for IgG1 (FIG. 11C) and IgG2a (FIG. 11D) to determine the isotype of the resulting antibody. Regardless of the delivery system used, the level of IgG2a was overwhelmingly higher than that of IgG1, indicating that Th1 type of antibody dominates the response.

### Experimental Example 10. Immune from the Ileum's Peyer's Patch

Dendritic cells, located extensively in the intestinal lamina propria, especially the Peyer's patch, play an important role in sampling and processing to present luminal antigens to T cells. To determine specific immune cells that interact with the antigen in vivo, the present inventors have isolated dendritic cells from the Peyer's patch of the ileum. The population of dendritic cells was analyzed by multicolor flow cytometry using a combination of markers (CD11c and MHC-II). After gating, the major dendritic cell populations expressing CD11c and a compatibility complex (MHC) class II were identified.

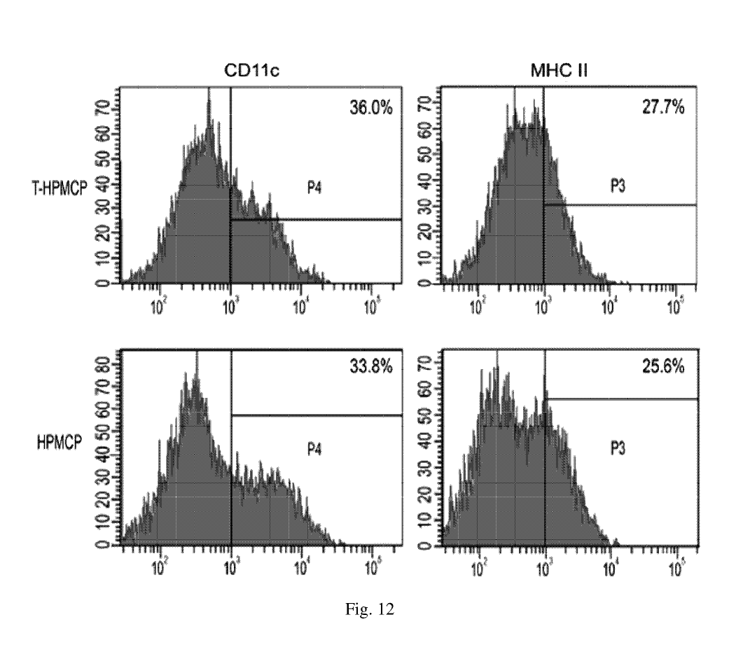

Mice administered with M-BmpB/THPMCP MP exhibited increased positive CD11c (36.0%) and MHC-II (27.7%) dendritic cell populations. On the other hand, the mice to which M-BmpB/HPMCP MP was administered exhibited dendritic cell populations with an increase of 33.8% of CD11c and 25.6% of MHC-II (FIG. 12).

### Experimental Example 11. Up-Regulated CD4⁺ T Cells in the Spleen of Immunized Mice

The final response of effective antigen delivery is the memory cell, which causes the accumulation of immune cells in the spleen. This is for future defense as adaptive immunity. To determine the efficiency of protein antigens to induce specific T-cell responses, splenocytes were isolated from mice immunized with antigen and stimulated in vitro with a cell stimulation cocktail.

Cells were analyzed by staining intracellular cytokines of CD4+, IFN-γ and IL-4 and performing flow cytometric analysis. Intracellular detection of IFN-γ and IL-4 in such spleen T lymphocytes can reveal the frequency of each cytokine producing cell and thus evaluate the persistence of cellular or humoral immune responses.

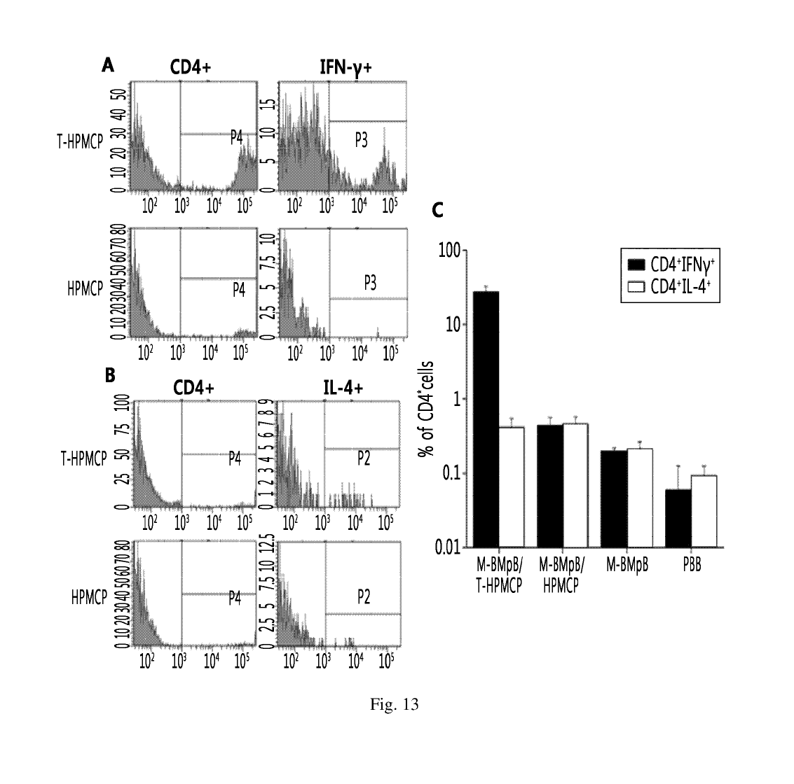

As a result, it was confirmed that the administration of M-BmpB/T-HPMCP MP resulted in a sharp increase of antigen-specific CD4⁺ T cells producing IFN-γ than in the case of antigen administration by control group or HPMCP MP. In particular, it was confirmed that there was no significant difference in the ratio of CD4⁺ T cells secreting IL-4 between different immunized groups or control groups (FIG. 13).

### Experimental Example 12. Preparation of Thiolated HPMCP Using Glutathione and Cysteamine

The present inventors sought to confirm whether the improved mucoadhesiveness of the thiolated HPMCP identified in the above Experimental Example is maintained in thiolated HPMCP prepared by other methods.

Specifically, thiolated HPMCP was prepared using glutathione and cysteamine as described below.

### Experimental Example 12-1. Synthesis of HPMCP-Glutathione Polymer Delivery Vehicle

HPMCP-Glutathione was prepared by dissolving HPMCP 55 (4 g) in 60 ml of dimethylsulfoxide (DMSO) as an organic solvent and then adding the activation agents, N—N′-dicyclohexylcarbodiimide (DCC) (4.87 g)/N-hydroxyl succinimide (NHS) (2.71 g) were dissolved in 30 ml of DMSO and 15 ml of DMSO, respectively, and the mixture was reacted with HPMCP at room temperature for 24 hours to activate the carboxyl group of HPMCP.

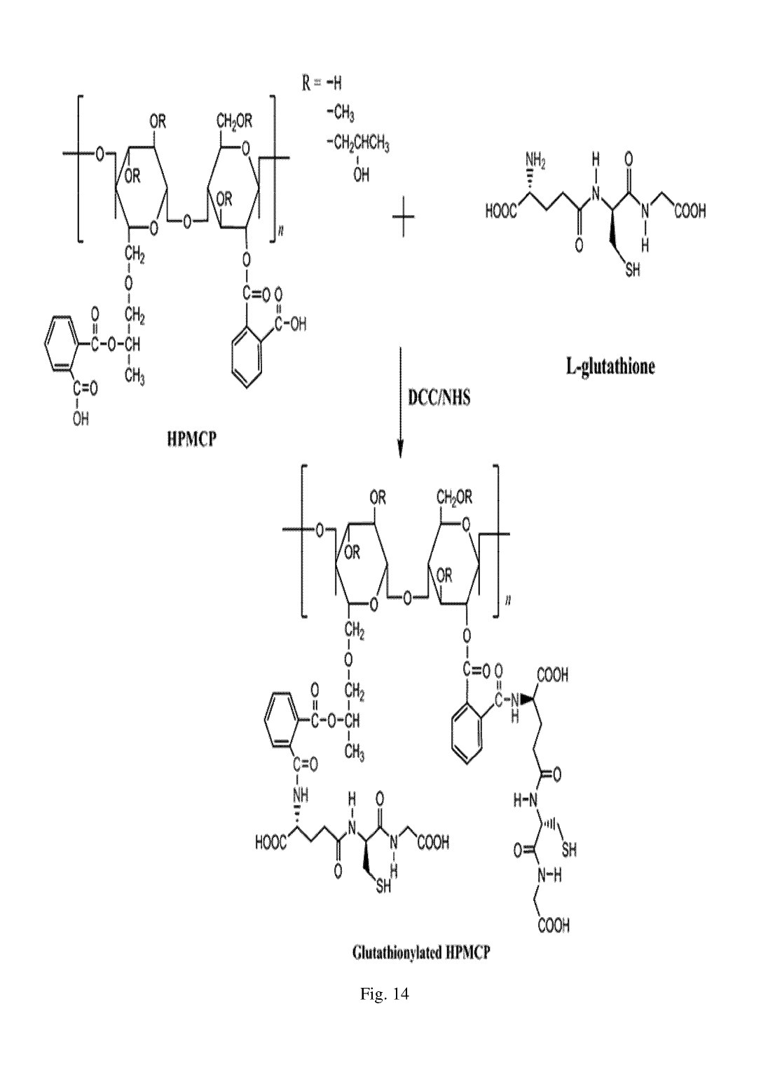

Then, glutathione (0.725 g) dissolved in DMSO was added and reacted for 48 hours to induce amide bond between HPMCP and L-glutathione (FIG. 14). In order to eliminate the unnecessary reaction by oxygen, each process was conducted under a nitrogen gas supply.

For removal of unreacted glutathione, 4 L of DMSO was dialyzed, and remaining DMSO was removed by dialysis several times for 3 days in 4 L of distilled water (D.W.). HPMCP-glutathione synthesized after completion of dialysis was obtained as a powder through lyophilization.

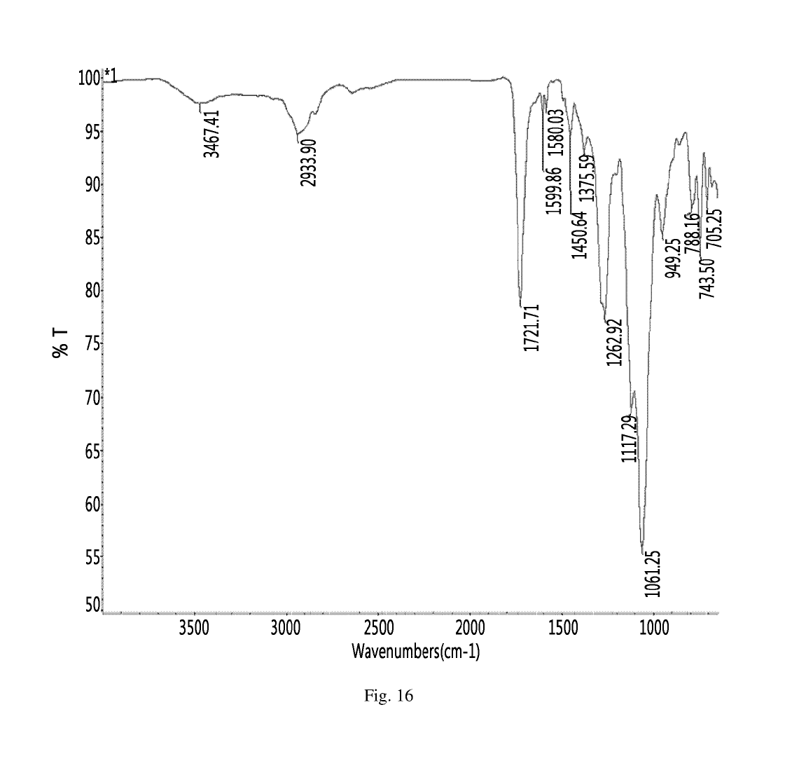

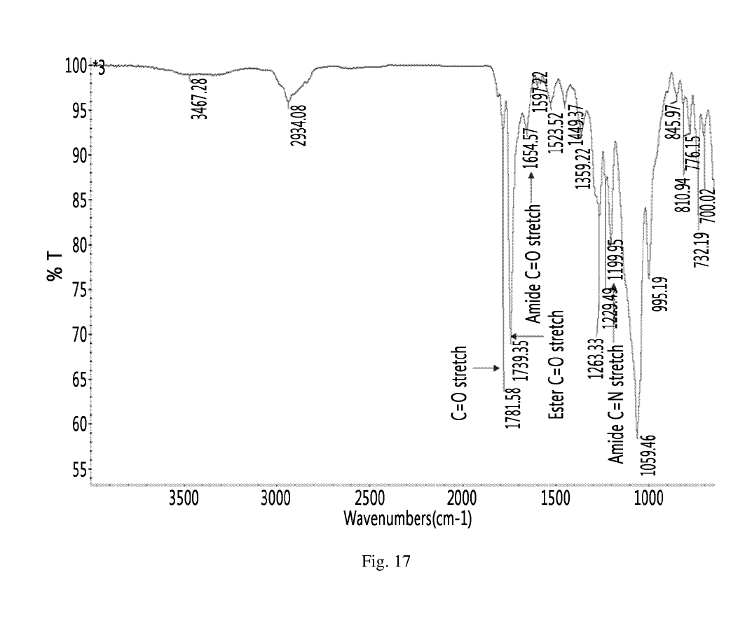

The synthesized HPMCP-glutathione polymer was verified by FTIR (Fourier transform infrared spectroscopy) (FIGS. 16 and 17).

### Experimental Example 12-2. Synthesis of HPMCP-Cysteamine Polymer Delivery Vehicle

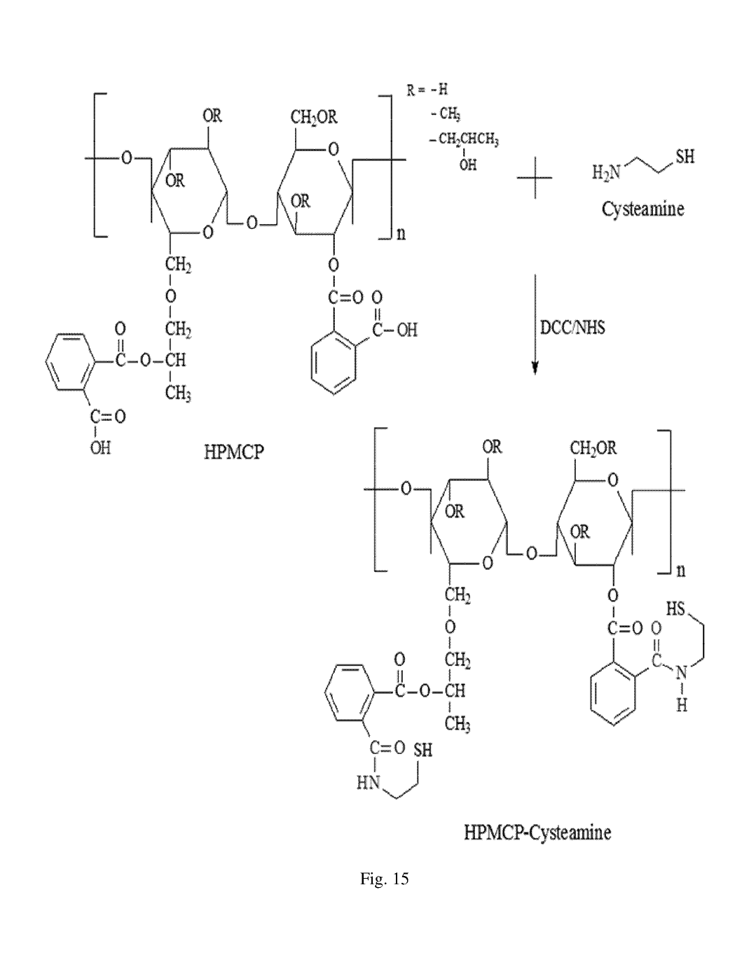

HPMCP-cysteamine was prepared by dissolving HPMCP 55 (4 g) in 60 ml of DMSO (organic solvent), dissolving the activation agents N,N′-dicyclohexylcarbodiimide (DCC) (4.87 g) and N-hydroxyl succinimide (NHS) (2.71 g) in each of 30 ml of DMSO and 15 ml of DMSO, and was reacted with HPMCP at room temperature for 24 hours to activate the carboxyl group of HPMCP. Then, cysteamine (0.268 g) dissolved in DMSO was added and reacted for 48 hours to induce amide bond between HPMCP and cysteamine (FIG. 15). In order to eliminate the unnecessary reaction by oxygen, each process was conducted under a nitrogen gas supply.

For removal of unreacted cysteamine, 4 L of DMSO was dialyzed, and the remaining DMSO was extracted by dialysis 4 L of distilled water (D.W.) several times for 3 days. HPMCP-cysteamine synthesized after completion of dialysis was obtained as a powder through lyophilization.

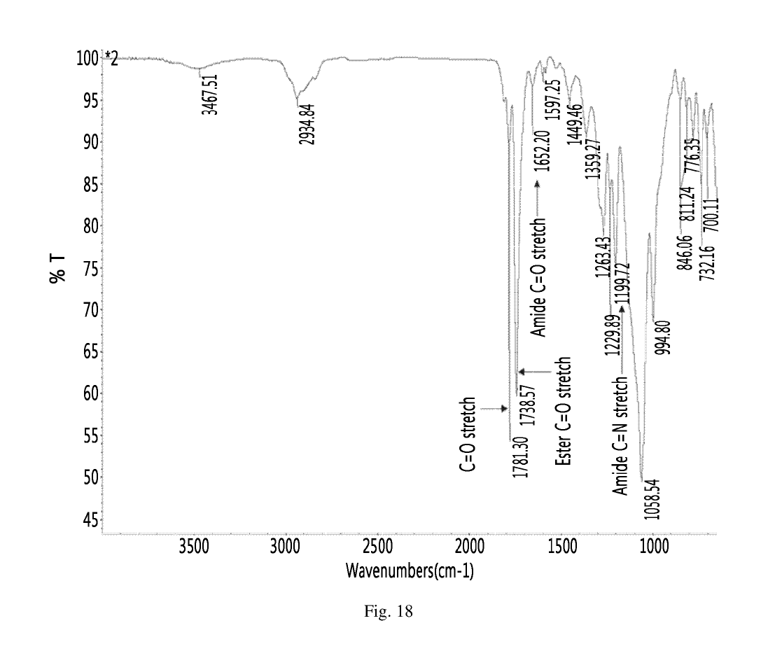

The synthesized HPMCP-cysteamine polymer was verified by FTIR (Fourier transform infrared spectroscopy) (FIGS. 16 and 18).

### Experimental Example 13. Confirmation of Mucoadhesiveness of Thiolated HPMCP Using Glutathione and Cysteamine

The present inventors tried to confirm the improved mucoadhesiveness of the thiolated HPMCP prepared in Experimental Example 12 above.

### Experimental Example 13-1. Bonding of HPMCP, HPMCP-Glutathione, HPMCP-Cysteamine Polymer Delivery Vehicle and FITC (Fluorescein Isothiocyanate)

HPMCP and 100 mg of each of HPMCP-glutathione and HPMCP-cysteamine prepared in Experimental Example 12 were dissolved in 3 ml of DMSO and stirring was performed in a dark room environment. 5 mg of FITC was dissolved in 0.1 ml of DMSO and reacted with stirring with HPMCP, HPMCP-glutathione, and HPMCP-cysteamine polymer delivery vehicle for 4 hours at room temperature. The reaction products were then dialyzed against DW for 24 hours and lyophilized.

### Experimental Example 13-2. Measurement of Mucoadhesiveness of HPMCP, HPMCP-Glutathione, and HPMCP-Cysteamine Polymer Delivery Vehicle

10 mg of FITC-conjugated HPMCP, HPMCP-glutathione and HPMCP-cysteamine are dissolved in 0.1 N NaOH solution. 2 mg of BmpB protein was dissolved in 0.2 ml of PBS and mixed with a polymer delivery vehicle dissolved in NaOH solution.

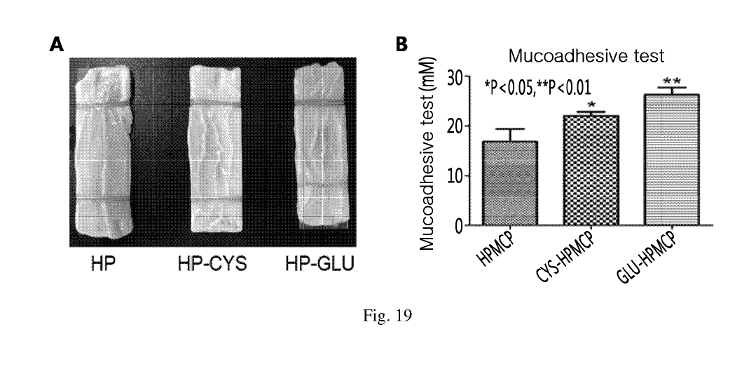

0.1 N of HCl was slowly dropped into the mixture to precipitate the nanoparticles. The obtained nanoparticles are suspended in 40 ml of PBS and transferred to a 50 ml falcon tube. Absorbance was measured at 495 nm before transfer, and a glass slide with a swine small intestine section slice was placed in a falcon tube. After culturing at 37° C. and 50 rpm for 1 hour, the absorbance was measured again at 495 nm (FIG. 19A).

When the nanoparticles were adhered to the small intestine, the absorbance of FITC in the solution was different. Therefore, the nanoparticles adhered to the small intestine mucosa were converted to evaluate the mucoadhesive ability of each polymer using the difference.

As a result, it has been confirmed that higher mucoadhesive ability was exhibited in the order of HPMCP-glutathione, HPMCP-cysteamine and HPMCP, and HPMCP-glutathione exhibited about 1.5 times higher mucoadhesive ability than unthiolated HPMCP (FIG. 19B).

From the above description, it will be understood by those skilled in the art that the present disclosure may be embodied in other specific forms without departing from the spirit or essential characteristics thereof. In this regard, it should be understood that the above-described embodiments are to be considered in all respects as illustrative and not restrictive. The scope of the present disclosure should be construed as being included in the meanings and scope of the appended claims rather than the detailed description and in all modifications or modified forms derived from their equivalents.

1. A thiolated hydroxypropyl methylcellulose phthalate (T-HPMCP) drug delivery vehicle which is ileum-specific pH responsive and is loaded with either a protein drug or an antigen.

2. The drug delivery vehicle according to claim 1, wherein the hydroxypropyl methylcellulose phthalate drug delivery vehicle is thiolated by introducing a thiol group from L-cysteine, glutathione, or cysteamine.

3. The drug delivery vehicle according to claim 1, wherein the drug delivery vehicle is dissolved at a pH of 7.4 or higher.

4. The drug delivery vehicle according to claim 1, wherein the antigen is M-BmpB.

5. The drug delivery vehicle according to claim 1, wherein the drug delivery vehicle is 1.5 times more mucoadhesive than the non-thiolated HPMCP.

6. The drug delivery vehicle according to claim 1, wherein the drug delivery vehicle remains in the mucosa at 50% or more after 2 hours of administration.

7. The drug delivery vehicle according to claim 1, wherein the drug delivery vehicle stimulates CD4⁺ T cells to induce adaptive immunity.

8. The drug delivery vehicle according to claim 7, wherein the CD4⁺ T cells produce interferon (IFN)-γ.

9. A method for producing T-HPMCP microparticles, comprising a step of homogenizing the thiolated hydroxypropyl methylcellulose phthalate (T-HPMCP) in the presence of an organic solvent.

10. The method for producing T-HPMCP microparticles according to claim 9, wherein the organic solvent is methane chloride.

11. The method for producing T-HPMCP microparticles according to claim 9, wherein the organic solvent is dichloromethane, a mixed solvent of dichloromethane and ethanol, or a mixed solvent of dichloromethane and methanol.

12. A method for producing a T-HPMCP drug delivery vehicle which is ileum-specific pH responsive, comprising a step of loading a T-HPMCP microparticle prepared by the method of claim 9 with a protein drug or an antigen.

13. A method for producing a T-HPMCP drug delivery vehicle which is ileum-specific pH responsive, comprising a step of loading a T-HPMCP microparticle prepared by the method of claim 10 with a protein drug or an antigen.

14. A method for producing a T-HPMCP drug delivery vehicle which is ileum-specific pH responsive, comprising a step of loading a T-HPMCP microparticle prepared by the method of claim 11 with a protein drug or an antigen.


SEQUENCE LISTING (1 sequence)

SEQ ID NO: 1 - protein, 9 aa, Brachyspira hyodysenteriae
CKSTHPLSC